(19) United States
(12) Patent Application Publication
(10) Pub. No.: US 2016/0193264 A1
(43) Pub. Date: Jul. 7, 2016

(54) COMPOSITION COMPRISING NETTLE

(71) Applicant: PANDELA S.R.L., Milano (IT)

(72) Inventor: Maria Oliva SALVIATI, MILANO (IT)

(21) Appl. No.: 14/392,227

(22) PCT Filed: May 9, 2014
(86) PCT No.: PCT/IB2014/061337

§ 371 Date: Dec. 24, 2015

(30) Foreign Application Priority Data
Jun. 25, 2013 (IT) MI 2013 A 001053

(51) Int. Cl. A61K 36/185; A61K 35/36; A61K 36/63; A61K 35/33

(52) U.S. Cl. CPC A61K 36/185; A61K 35/33; A61K 35/36; A61K 36/63

(57) ABSTRACT

The present invention provides compositions which include one or more oils and Oleum coctus Urticae. The present invention also provides methods for preparing such compositions and treatment and/or prevention of skin diseases and physiological degenerative processes of the skin in mammals by using such compositions alone or in combination with excipients and as a co-adjuvant in combination with other therapies.

The present invention relates to a composition comprising one or more oils and Oleum coctus Urticae.

A further object of the present invention is a method of preparing said composition and the use thereof in the treatment and/or prevention of skin diseases and of physiological degenerative processes of the skin in mammals.

### BACKGROUND OF THE ART

There are hundreds of skin diseases, and some of them may present with apparently similar or overlapping clinical manifestations.

The group encompasses, inter alia, skin and subcutaneous tissue infections, bullous diseases, dermatitis and eczema, papulosquamous disorders, urticaria and erythema, photodermatoses and skin changes by ionizing radiations, pigmentation disorders, decubitus ulcer, fibromatosis, lupus erythematosus, skin disorders during vasculitis and other angiopathies, skin malignancies, benign skin tumors.

In addition to manifestations of a pathological type, skin incurs degenerative processes of a physiological type, related to age progression.

The need is strongly felt, of compositions capable of recovering the skin degenerations, whether they are of pathological or a physiological type.

Unguents, composed of a low water content base, are indicated in the treatment of dry, squamous dermatoses. In fact, the fat base, by virtue of its occlusive properties, exerts an advantageous wetting effect, allowing a deeper action of the medicament; the unguents are particularly suitable to skin conditions that may benefit from a prolonged massage and are characterized by a slow absorption. Oil and wax-base unguents are known, where from 5 to 7 parts of oil or oil mixture are combined to one part of bee wax. Unguents are also known, where from 7 to 10 parts of oil or oil mixture are combined with one part of vegetal wax.

Nettle, which is a plant of the genus Urtica, in addition to the known allergic skin reaction, is known in pharmacognosis for its antiphlogistic, diuretic, prostatic properties.

The nettle fruits contain oils, mucilages, and proteins; the aerial parts contain flavonoids, vitamins, chlorophyll, mineral salts, and secretin; its stinging hairs contain histamine, acetylcholine, and formic acid, which is responsible for the stinging effect. Finally, nettle root contains phenols, tannin, sterols, lecithin, and polysaccharides.

Fats contained in the seeds are provided with restorative properties and are used in cases of asthenia; recent studies showed that the nettle root has beneficial effects in case of prostatic hypertrophy, resulting in a reduction in the gland volume. Also a diuretic and anti-inflammatory action is associated with the same root. The nettle leaves, due to the presence of mineral salts and chlorophyll, would have an anti-anaemic action. The nettle juice invigorates and stimulates the scalp. It is used against seborrhea and hair loss.

Hair care nettle-based shampoos are currently available, in addition to formulations comprising nettle for internal use.

### DESCRIPTION OF THE INVENTION

The present invention discloses a composition comprising one or more oils and Oleum coctus Urticae.

Said composition finds use in the prevention and/or in treatment of skin diseases and physiological degenerative processes of the skin in mammals.

### DETAILED DESCRIPTION OF THE INVENTION

The present invention discloses a composition comprising one or more oils and Oleum coctus Urticae.

In a preferred embodiment, said composition comprises, in addition, one or more waxes.

By nettle is meant herein the plant of the genus Urtica, in one of the dioica, urens, or pilulifera species. To the purposes of the present invention, the raw plant, aerial part, and/or roots are used.

By the term Oleum coctus Urticae is meant herein the nettle extract obtained by immersing the raw plant, aerial part, and/or roots, in hot oil. The plant is macerated during a few minutes; subsequently, one proceeds with filtration to remove the plant residues.

Said composition comprises, per each liter of oil or oil mixture, Oleum coctus Urticae obtained from 1-500 g of raw plant, aerial part, and/or roots of nettle, or 10 to 250 g, or 15 to 185 g, or 20 to 100 g, or 25 to 60 g, or 30 to 50 g, preferably from 35 g of raw plant, aerial part, and/or roots of nettle.

In a preferred embodiment, where said composition comprises also one or more waxes, said composition comprises, per one part of wax or wax mixture, 7 to 25 parts by weight of oil, or oil mixture, and Oleum coctus Urticae obtained from 0.10-3 parts by weight of raw plant, aerial part, and/or roots of nettle. Preferably, said composition comprises from 8 to 20 parts of oil or oil mixture and Oleum coctus Urticae from 0.30-2 parts by weight of nettle, or from 10 to 15 parts of oil or oil mixture and Oleum coctus Urticae from 0.40-1 part of nettle. In a preferred embodiment, said composition comprises about 11.4 parts of oil or oil mixture and Oleum coctus Urticae from about 0.44 parts by weight of nettle per one part of wax.

Said one or more oils are preferably vegetal oils, selected, by way of non-limiting example, from argan oil, almond oil, rose hip oil, shea oil, chamomile oil, avocado oil, lemon oil, wheat germ oil, carrot oil, calendola oil, oil of Hypericum, ivy, coconut, macassar, hazelnut, pomegranate, lavender cotton, Helichrysum, baobab, apricot, sesame, sunflower, walnut, corn, olive. In a preferred embodiment, said oil is olive oil.

Said one or more waxes are synthetic or natural waxes. By way of example, said synthetic wax may be isopropyl myristate. Preferably, they are one or more natural waxes, selected from waxes of vegetal origin, such as, by way of example, carnauba wax and Jojoba oil, or of animal origin, such as, by way of example, bee wax, spermaceti, uropigyal ester, and lanolin. In a preferred embodiment, said wax is bee wax.

The composition of the present invention may be comprised in a dermatological/dermal-cosmetic preparation that may comprise; in addition, suitable pharmaceutically acceptable excipients, such as antioxidant substances, for example Vitamin E, gelling substances, emulsifiers, diluents, or other additives known in the pharmaceutics art.

Said composition may further comprise one or more active ingredients of natural or synthetic origin.

In a further embodiment, said composition will be able to comprise a cell suspension representative of one or more epidermal cell types. By way of example, a cell suspension will be able to be used, comprising undifferentiated basal cells and/or keratinocytes and/or melanocytes and/or fibroblasts and/or Langerhans cells. Said cells will be heterologous cells, or preferably, they will be autologous cells.

The composition of the present invention is prepared according to the following procedure. The oil or oil mixture is heated at a temperature ranging between 140 and 180° C., preferably about 160° C. The raw plant, aerial part, and/or roots of nettle are then added to the hot oil, and it is left macerating from at least 1 minute up to 3 hours, or up to 1 hour, or up to 40 minutes, or up to 20 minutes, or up to 10 minutes. Preferably, maceration is carried out during about 3 minutes. Then, one proceeds with a filtration so as to remove the plant residues, and the filtrate is the composition of the present invention.

Where said composition also comprises one or more waxes, the wax or wax mixture is added to the preparation at the end of said maceration step. The composition is suitably mixed to melt the wax or wax mixture; then, a filtration is carried out. The filtrate is the composition at issue.

It is a further aspect of the present invention a method of preparing extracts from raw plants, comprising the following steps:
an oil or oil mixture is heated at a temperature ranging between 140 and 180° C., preferably about 160° C.;
a raw plant, aerial part, and/or roots is added to said hot oil or oil mixture, which is left macerating from at least 1 minute up to 3 hours, or up to 1 hour, or up to 40 minutes, or up to 20 minutes, or up to 10 minutes; preferably, maceration is carried out during about 3 minutes;
the mixture is filtered, and the filtrate is the extract.

Said extract has unique characteristics in terms of active ingredients extracted compared to extracts obtained by method that are known in the state of the art; furthermore, said extract has the characteristic of a prolonged stability, even in non-optimal environments conditions.

The composition of the present invention finds application in treating or prevention of cutaneous degenerative phenomena, of a pathological or physiological type, in a mammal, preferably humans, dog, cat, horses, pigs, and cattle.

Said composition is used, by way of non-limiting example only, in the treatment of skin and subcutaneous tissue infections, septic injuries, blast injuries, insect stings, bullous diseases, dermatitis and eczema, papulosquamous disorders, urticaria and erythema, photodermatoses and skin changes by ionizing radiations, pigmentation disorders, decubitus ulcer, fibromatosis, lupus erythematosus, skin disorders during vasculitis and other angiopathies, skin malignancies, benign skin tumors, skin disorders of psoriasis. Said composition finds also application in skin complications due to treatment with corticoids, cytostatic agents, retinoic acid and derivatives thereof, or with other natural substances and/or synthetic substances. In particular, said composition is useful in the treatment of I, II, III, and IV degree burns, atopic dermatitis, skin disorders of psoriatic arthritis, in the treatment of phlyctenae, tyloma, skin ulcers, decubitus ulcers. Said composition finds also use in vascular diseases conditions, such as trophic ulcer, diabetes ulcer, skin vascular necrosis, anal rhagades, hemorrhoids, ulcers, or phlogosis of vaginal mucosae.

Said composition finds also use as a co-adjuvant in therapy. Preferably, said therapy is selected from therapies providing for the use of laser or of ultraviolet radiations or photochemotherapies, surgeries, chemotherapy, radiotherapy, treatments with corticoids and/or other natural and/or synthetic substances for topic use. When used as a co-adjuvant, the application may be carried out concomitantly with or delayed with respect to the further treatment, according to the subject's needs.

The composition of the present invention finds further use in the prevention and in treatment of skin aging.

The frequency of application of said composition or the dermatological/dermal-cosmetic preparation comprising it varies according to the subject's needs. Said frequency may range between once per month and 10 times a day, preferably between once a week and 4 times a day, more preferably between 3 times a week and 3 times a day, still more preferably 1 or 2 times a day.

As best illustrated in the following examples, the effects obtained by the use of the composition of the present invention are surprising.

Said composition, used for the treatment of first degree burns, results in the disappearance of pain within minutes. When it is used for the treatment of second and third degree burns, it results in a complete remission of pain within 12/24 hours. In addition, subsequent the inflammation and infection sensibly decrease within 48 hours from the treatment until reaching a full healing.

The use of said composition in trophic ulcers, also known by the name of varicose ulcers, results, within 24 hours, in a recovery of about 20% and, within 15 days from the beginning of the treatment, recovery is of about 70%.

Surprisingly, when it is used in the treatment of advanced-stage decubitus ulcers, already purulent, said composition results in a recovery of about 50% after 30 days of treatment and the recovery is almost complete after 2 months of treatment.

### DESCRIPTION OF THE FIGURES

FIG. 1: painful trophic ulcer of V metatarsal before treatment.

FIG. 2: painful trophic ulcer of the V metatarsal after 24 hours and after 15 days of treatment.

FIG. 3: necrotic vascular trophic ulcer before treatment.

FIG. 4: necrotic vascular trophic ulcer after 10, 28 days and 3 months of treatment.

FIG. 5: decubitus ulcer before treatment.

FIG. 6: decubitus ulcer after 30, 40, 50, and 60 days of treatment.

FIG. 7: ulcer due to plaster cast device.

FIG. 8: ulcer due to plaster. cast device after 30 days of treatment.

### EXAMPLES

### Example 1

### Preparation of a Composition Comprising Olive Oil, Bee Wax, and Oleum coctus Urticae

1 liter olive oil is heated to a temperature of about 160° C. When the oil has reached the temperature indicated, 35 g of nettle, the whole raw plant, is added, and left during about 3 minutes. At the end, 80 g bee wax is added to the preparation. The preparation is mixed so as to dissolve the bee wax; subsequently, a filtration is carried out, thus obtaining a filtrate that, once cooled, is the ready-to-use composition.

### Example 2

### Use of a Composition Comprising Oleum coctus Urticae in the Treatment of a Vascular Trophic Ulcer of the V Metatarsal Bone

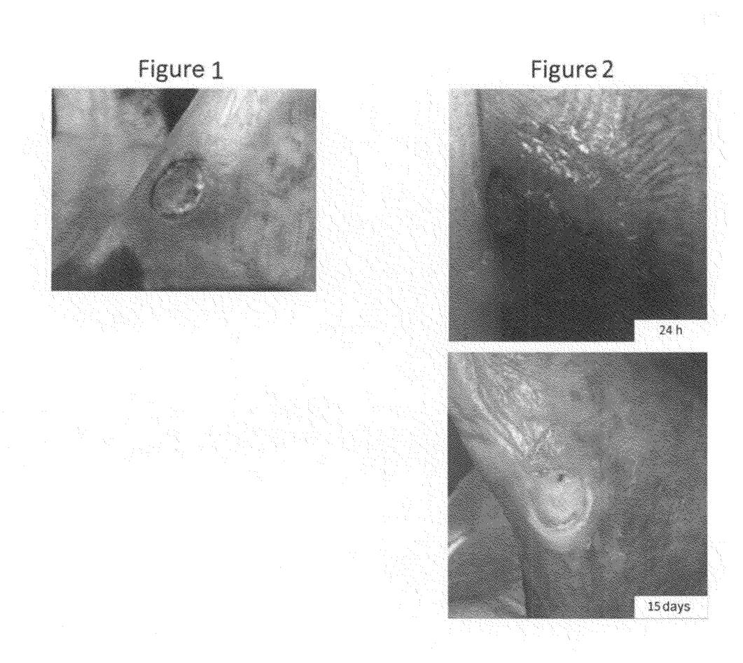

A composition prepared according to the example 1 is applied onto the area affected from a painful vascular trophic ulcer of the V metatarsal, with appearance as in FIG. 1. In the following days, one proceeds with the application of the same composition twice a day. 24 hours after the first treatment, pain is absent. On day 7 of treatment, the ulcer appears as in FIG. 2. On day 15 of treatment, the ulcer is clinically healed, as shown by the image set forth in the same FIG. 2.

### Example 3

### Use of a Composition Comprising Oleum coctus Urticae in the Treatment of a Necrotic Vascular Trophic Ulcer

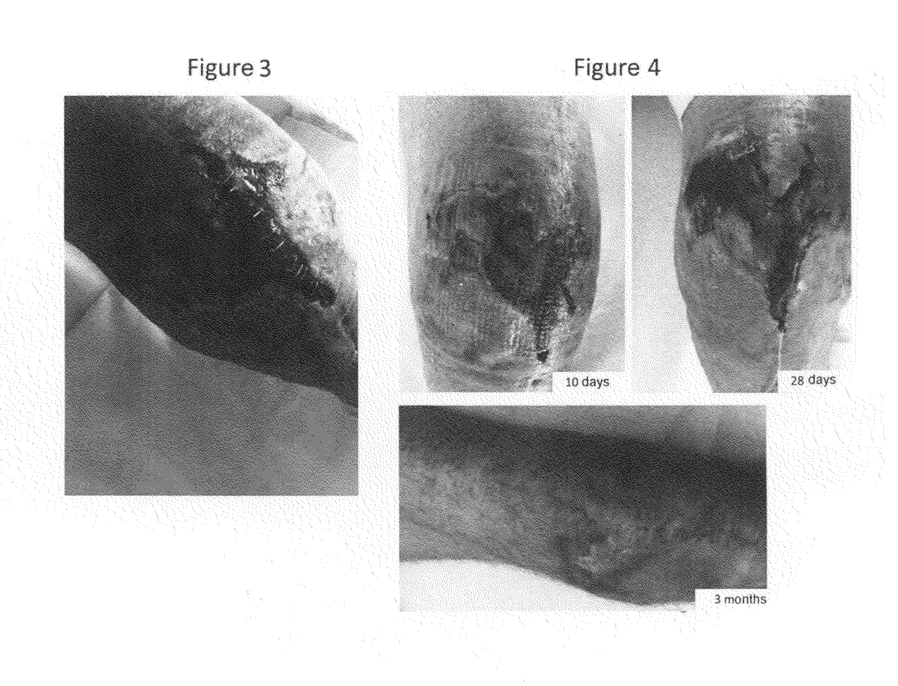

A diabetic subject with vascular disease has an infected necrotic vascular trophic ulcer on a kneel prosthesis. Said ulcer is, before the treatment, as illustrated in FIG. 3. The composition prepared as described in the example 1 is applied on the part at issue. During the following days, one proceeds with the application of the same composition twice a day. On day 10, a recovery can be noticed, as shown in FIG. 4. Said recovery is consistent on day 28 of treatment, FIG. 4. At 3 months, the recovery is clear, FIG. 4.

### Example 4

### Use of a Composition Comprising Oleum coctus Urticae in the Treatment of a Decubitus Ulcer

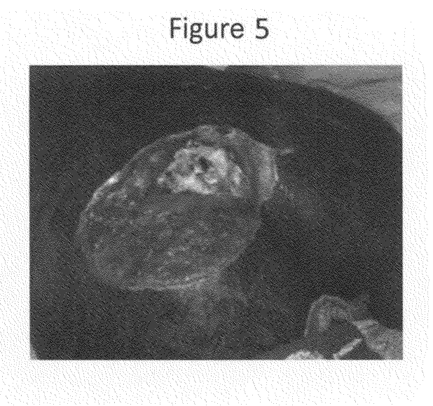

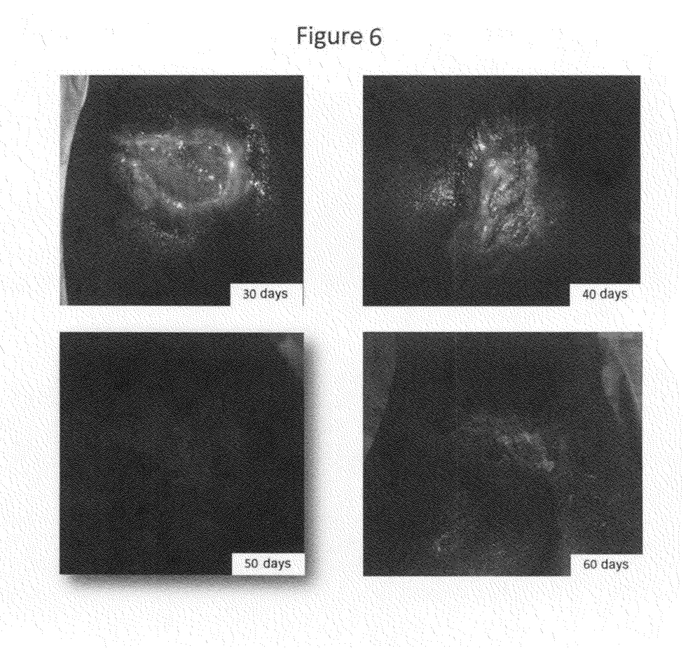

A 12-years old child affected from spastic palsy, has decubitus ulcers with substantial dimensions, as illustrated in FIG. 5. The composition prepared as described in the example 1 is applied on the part at issue, and the treatment is repeated during the next 60 days, with applications twice a day. FIG. 6 sets forth the recovery noticed at 30, 40, 50 and 60 days. Surprisingly, recovery is complete within day 60.

### Example 5

### Use of a Composition Comprising Oleum coctus Urticae in the Treatment of an Ulcer Due to a Plaster Cast Device

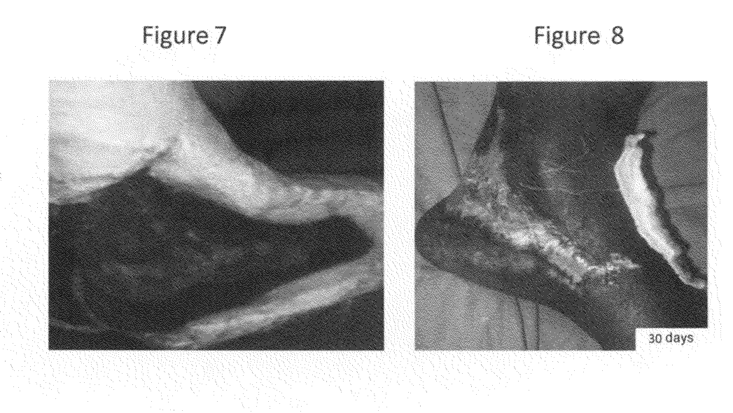

An ulcer due to a plaster cast device as illustrated in FIG. 7 is treated with the composition prepared as described in the example 1. The treatment is repeated during the next 60 days, with applications twice a day. FIG. 8 sets forth the recovery noticed at 30 and 60 days. Surprisingly, recovery is complete within day 60.

1-21. (canceled)

22. A composition comprising one or more oils and Oleum coctus Urticae.

23. The composition of claim 22 comprising, per each liter of oil or oil mixture, Oleum coctus Urticae obtained from 1-500 g of raw plant, aerial part, and/or roots of nettle.

24. The composition of claim 22, further comprising one or more waxes.

25. The composition of claim 24, comprising, per one part of wax or wax mixture, 7 to 25 parts by weight of oil or oil mixture and Oleum coctus Urticae obtained from 0.10-3 parts by weight of raw plant, aerial part, and/or roots of nettle.

26. The composition of claim 24, comprising, per one part of wax or wax mixture, about 11.4 parts of oil or oil mixture and Oleum coctus Urticae obtained from about 0.44 parts by weight of raw plant, aerial part, and/or roots of nettle.

27. The composition of claim 22, wherein said one or more oils comprise vegetable oils selected from the group consisting of: argan oil, almond oil, rose hip oil, shea oil, chamomile oil, avocado oil, lemon oil, wheat germ oil, carrot oil, calendola oil, oil of Hypericum, ivy, coconut, macassar, hazelnut, pomegranate, lavender cotton, Helichrysum, baobab, apricot, sesame, sunflower, walnut, corn, and olive.

28. The composition of claim 22, wherein said oil comprises olive oil.

29. The composition of claim 24, wherein said one or more waxes comprise synthetic or natural waxes, of vegetable origin or animal origin.

30. The composition of claim 24, wherein said wax comprises beeswax.

31. The composition of claim 22, further comprising one or more active ingredients of natural or synthetic origin.

32. The composition of claim 22, further comprising a cell suspension representative of one or more epidermal cell types, wherein said cells are selected from the group consisting of: undifferentiated basal cells, keratinocytes, melanocytes, fibroblasts, and Langerhans cells.

33. A method of preparing a composition from extracts of raw plants, said method comprising:
heating one or more oils at a temperature from 140 to 180° C.;
adding raw plant, aerial part, and/or roots of nettle;
macerating from at least 1 minute up to 3 hours, and
filtering, to obtain said composition.

34. The method of claim 33, wherein said plant is nettle and said extract is a composition comprising one or more oils and Oleum coctus Urticae.

35. The method of claim 33, further comprising, after said maceration step and before carrying out said filtration:
adding one or more waxes; and
mixing the composition.

36. The method of claim 33, wherein said one or more oils comprise vegetable oils selected from the group consisting of: argan oil, almond oil, rose hip oil, shea oil, chamomile oil, avocado oil, lemon oil, wheat germ oil, carrot oil, calendola oil, Hypericum oil, oil of ivy, of coconut, macassar, hazelnut, pomegranate, lavender cotton, Helichrysum, baobab, apricot, sesame, sunflower, walnut, corn, and olive.

37. The method of claim 35, wherein said one or more waxes comprise synthetic or natural waxes, of vegetable origin or animal origin.

38. A method for treating or preventing cutaneous degenerative conditions of a pathological or physiological type in a mammal, comprising administering the composition of claim 22 to a mammal in need thereof.

39. The method of claim 38, wherein said cutaneous degenerating condition is selected from the group consisting of: skin and subcutaneous tissue infections, septic injuries, blast injuries, insect stings, bullous diseases, dermatitis and eczema, papulosquamous disorders, urticaria and erythema, photodermatoses and skin changes by ionizing radiations, pigmentation disorders, decubitus ulcer, fibromatosis, lupus erythematosus, skin disorders during vasculitis and other angiopathies, skin malignancies, benign skin tumors, skin disorders of psoriasis; skin complications due to treatment with corticoids, cytostatic agents, retinoic acid and derivatives thereof, or with other natural substances and/or synthetic substances, I, II, III, and IV degree burns, atopic dermatitis, skin disorders of psoriatic arthritis, phlyctenae, tyloma, trophic ulcer, diabetes ulcer, skin vascular necrosis, anal rhagades, hemorrhoids, ulcers and/or phlogosis of vaginal mucosae, and skin aging.

40. A method for treating or preventing cutaneous degenerative conditions of a pathological or physiological type in a mammal, comprising administering the composition of claim 22 as a co-adjuvant in therapy, wherein said therapy is selected from the group consisting of a therapy using laser or ultraviolet radiation or photochemotherapies, surgeries, chemotherapy, radiotherapy, treatment with corticoids and/or other natural and/or synthetic substances for topic use.

41. A dermatological/dermal-cosmetic preparation comprising the composition of claim 22 and pharmaceutically acceptable excipients.
